(19) United States
(12) Patent Application Publication
(10) Pub. No.: US 2019/0209030 A1
(43) Pub. Date: Jul. 11, 2019

(54) BLOOD PRESSURE STATUS MEASURING APPARATUS

(71) Applicant: Murata Manufacturing Co., Ltd., Nagaokakyo-shi (JP)

(72) Inventor: Toru Shimuta, Nagaokakyo-shi (JP)

(21) Appl. No.: 16/263,428

(22) Filed: Jan. 31, 2019

Related U.S. Application Data
(63) Continuation of application No. PCT/JP2017/028703, filed on Aug. 8, 2017.

(30) Foreign Application Priority Data
Aug. 10, 2016 (JP) 2016-157257

(51) Int. Cl. A61B 5/021; A61B 5/024; A61B 5/0245; A61B 5/0456; A61B 7/02; A61B 5/00

(52) U.S. Cl. CPC A61B 5/02125; A61B 5/02416; A61B 5/0245; A61B 2562/0247; A61B 7/02; A61B 5/6822; A61B 5/0456

(57) ABSTRACT

A blood pressure status measuring apparatus includes a first photoplethysmographic sensor that includes a light emitting element) outputting near-infrared light and acquires a photoplethysmographic signal of a carotid artery, a second photoplethysmographic sensor that includes a light emitting element outputting blue to yellow-green light and acquires a photoplethysmographic signal of an arteriole or a capillary near the carotid artery, a pulse wave propagation time measuring unit that acquires a pulse wave propagation time on the basis of the photoplethysmographic signal of the carotid artery and the photoplethysmographic signal of the arteriole or the capillary near the carotid artery, a pulse wave propagation time change acquisition unit that acquires a temporal change of the pulse wave propagation time after measurement is started, and a blood pressure status measuring unit that measures circulatory dynamics including a blood pressure status on the basis of the temporal change of the pulse wave propagation time after measurement is started.

### CROSS REFERENCE TO RELATED APPLICATIONS

The present application is a continuation of International application No. PCT/JP2017/028703, filed Aug. 8, 2017, which claims priority to Japanese Patent Application No. 2016-157257, filed Aug. 10, 2016, the entire contents of each of which are incorporated herein by reference.

### TECHNICAL FIELD

The present invention relates to a blood pressure status measuring apparatus, and particularly, to a blood pressure status measuring apparatus using a pulse wave propagation time.

### BACKGROUND OF THE INVENTION

In recent years, for example, as an index for evaluation of the degree of arteriosclerosis, estimation of the life span of blood vessels, and the like, a pulse wave propagation time, which is a time in which a pulse wave propagates inside an artery of a living body (for example, a time up to arrival of a pulse wave from an R wave of an electrocardiogram), has been used. The pulse wave propagation time reflects a change in blood pressure.

A technique for calculating systolic blood pressure on the basis of a pulse wave propagation time (a non-invasive continuous blood pressure monitoring apparatus) is disclosed in Japanese Unexamined Patent Application Publication No. 2014-105 (Patent Document 1). In this technique, the pulse wave propagation time is calculated on the basis of a biological signal obtained by a biological signal detection sensor (a pulse wave detector (PPG detector) and an electrocardiographic detector (ECG detector)) that is worn on a subject, and systolic blood pressure is calculated on the basis of the acquired pulse wave propagation time and a blood pressure calculation expression. Furthermore, in this technique, a 3-axis acceleration detection sensor is worn on a subject so that posture and action of the subject can be detected from detection data, and blood pressure data and action data of the subject can be obtained at the same time in a chronological order. Therefore, a change in blood pressure, and posture and action can be monitored at the same time.

As described above, with the technique (non-invasive continuous blood pressure monitoring apparatus) of Patent Document 1, calculation of blood pressure based on an obtained pulse wave propagation time and detection of posture and action of a subject are performed at the same time. Therefore, a change in the blood pressure (a change in the pulse wave propagation time), and the posture and action of the subject can be monitored simultaneously.

It has been widely known that there is a high risk that arterial hypertension will lead to cerebral bleed and the like. In actuality, however, it is often the case that bleeding occurs in arterioles or capillaries that are narrower than arteries. Therefore, in order to estimate the risk of cerebrovascular diseases such as cerebral bleed, cardiovascular diseases, or the like, it is preferable to measure the blood pressure status or the like of arterioles or capillaries. However, in the technique of Patent Document 1, accurately measuring circulatory dynamics including the blood pressure status of arterioles or capillaries is not considered. Furthermore, in the technique of Patent Document 1, measuring circulatory dynamics including the blood pressure status in a more simplified way is not also considered.

### BRIEF SUMMARY OF THE INVENTION

The present invention has been made to solve the above problems, and an object of the present invention is to provide a blood pressure status measuring apparatus that is capable of measuring circulatory dynamics including the blood pressure status of arterioles or capillaries more simply and more accurately.

As a result of earnest research and development, it was found by the inventor that a change of a pulse wave propagation time of an arteriole or a capillary (an arteriole or the like) after measurement is started is correlated with a blood pressure status/circulatory dynamics of the corresponding part. Thus, a blood pressure status measuring apparatus according to one aspect of the present invention includes a photoplethysmographic sensor that includes a light emitting element and a light receiving element and acquires a photoplethysmographic signal of an arteriole or a capillary; a biological sensor that acquires a biological signal serving as a reference for measurement of a pulse wave propagation time; pulse wave propagation time acquiring means for acquiring a pulse wave propagation time on the basis of the photoplethysmographic signal of the arteriole or the capillary acquired by the photoplethysmographic sensor and a biological signal serving as a reference acquired by the biological sensor; change acquiring means for acquiring a temporal change of the pulse wave propagation time acquired by the pulse wave propagation time acquiring means after measurement is started; and measuring means for measuring circulatory dynamics including a blood pressure status on the basis of the temporal change of the pulse wave propagation time after the measurement is started acquired by the change acquiring means.

With the blood pressure status measuring apparatus according to the present invention, circulatory dynamics including the status of blood pressure of an arteriole or a capillary can be measured on the basis of a temporal change of a pulse wave propagation time obtained from a photoplethysmographic signal of the arteriole or the capillary after measurement is started (for example, the beginning of measurement). At this time, for example, by measuring the pulse wave propagation time between the arteriole or the capillary and an artery near the arteriole or the capillary and measuring a temporal change of the pulse wave propagation time at the beginning of measurement, a difference in blood pressure between the arteriole or the capillary and the artery can be measured, and circulatory dynamics can be measured. As a result, circulatory dynamics including the blood pressure status of the arteriole or the capillary can be measured more simply and more accurately.

In the blood pressure status measuring apparatus according to the present invention, preferably, the biological sensor is pulse wave detecting means for acquiring a pulse wave signal of an artery from which the arteriole or the capillary branches off, and the pulse wave propagation time acquiring means acquires a pulse wave propagation time on the basis of the photoplethysmographic signal of the arteriole or the capillary acquired by the photoplethysmographic sensor and the pulse wave signal of the artery acquired by the pulse wave detecting means.

In this case, the pulse wave propagation time between the arteriole or the capillary and the (nearby) artery from which the arteriole or the capillary branches off can be measured, and a temporal change of the pulse wave propagation time at the beginning of measurement can be measured. Therefore, a difference in blood pressure between the artery and the arteriole or the capillary can be measured, and circulatory dynamics can be measured. In this case, in particular, both the pulse wave propagation times (blood pressure etc.) can be measured at a single position. Therefore, blood pressure and the like of an arteriole or a capillary, including a wearing time, can be measured more simply. Furthermore, in this case, unlike measurement at separate two points (two positions), blood pressure and the like of an arteriole or a capillary can be measured genuinely (that is, accurately).

In the blood pressure status measuring apparatus according to the present invention, preferably, the light emitting element outputs blue to yellow-green light, and the pulse wave detecting means is a photoplethysmographic sensor that includes a light emitting element outputting near-infrared light.

Unlike near-infrared light (for example, light with a wavelength of 800 to 1000 nm), light of a visible light region (for example, blue to yellow-green light with a wavelength of 450 to 580 nm) is easily absorbed by a living body. Therefore, with the use of a light source of the visible light region (photoplethysmographic sensor) as a light emitting element (photoplethysmographic sensor), light is difficult to reach an artery underneath the skin. Thus, even if a photoplethysmographic sensor is arranged immediately above an artery, a photoplethysmographic signal of an arteriole or a capillary can be obtained. In contrast, with the use of a photoplethysmographic sensor that includes a light emitting element outputting near-infrared light that is relatively difficult to be absorbed by a living body, as the pulse wave detecting means, a photoplethysmographic signal of a carotid artery can be obtained. Accordingly, in this case, a photoplethysmographic signal corresponding to the blood flow of an arteriole or a capillary and a photoplethysmographic signal corresponding to the blood flow of a wider artery can be obtained at the same time in adjacent regions (that is, a pulse wave propagation time can be obtained).

In the blood pressure status measuring apparatus according to the present invention, preferably, the light emitting element outputs blue to yellow-green light, and the pulse wave detecting means is a piezoelectric pulse wave sensor that acquires a piezoelectric pulse wave signal.

As described above, unlike near-infrared light, light of a visible light region (in particular, blue to yellow-green with a wavelength of 450 to 580 nm) is easily absorbed by a living body. Therefore, with the use of a light source of the visible light region (photoplethysmographic sensor) as a light emitting element (photoplethysmographic sensor), light is difficult to reach an artery underneath the skin. Thus, even if a photoplethysmographic sensor is arranged immediately above an artery, a photoplethysmographic signal of an arteriole or a capillary can be obtained. In contrast, with the use of a piezoelectric pulse wave sensor using, for example, a piezoelectric element, a piezoelectric film, or the like, which mainly responds to pulsation of an artery, as the pulse wave detecting means, a pulse wave signal (piezoelectric pulse wave signal) of an artery can be obtained, without being much affected by pulsation of an arteriole or a capillary. Accordingly, in this case, a photoplethysmographic signal corresponding to the blood flow of an arteriole or a capillary and a piezoelectric pulse wave signal corresponding to the blood flow of a wider artery can be obtained at the same time in adjacent regions (that is, a pulse wave propagation time can be obtained).

In the blood pressure status measuring apparatus according to the present invention, preferably, the biological sensor is a sensor that acquires an electrocardiographic signal, and the pulse wave propagation time acquiring means acquires a pulse wave propagation time on the basis of the photoplethysmographic signal of the arteriole or the capillary acquired by the photoplethysmographic sensor and an R wave of the electrocardiographic signal acquired by the biological sensor.

In this case, the pulse wave propagation time between the heart and an arteriole or a capillary can be obtained on the basis of a photoplethysmographic signal of the arteriole or the capillary and an R wave (peak) of an electrocardiographic signal.

The blood pressure status measuring apparatus according to the present invention, preferably, further includes an electrocardiographic electrode that acquires an electrocardiographic signal. It is preferable that the pulse wave propagation time acquiring means acquires, in addition to the pulse wave propagation time based on the photoplethysmographic signal of the arteriole or the capillary acquired by the photoplethysmographic sensor and the pulse wave signal of the artery acquired by the pulse wave detecting means, a pulse wave propagation time based on the photoplethysmographic signal of the arteriole or the capillary acquired by the photoplethysmographic sensor and the R wave of the electrocardiographic signal acquired by the electrocardiographic electrode and a pulse wave propagation time based on the pulse wave signal of the artery acquired by the pulse wave detecting means and the R wave of the electrocardiographic signal acquired by the electrocardiographic electrode.

In this case, an electrocardiogram (in particular, an R wave) is measured at the same time as a reference. Thus, in addition to a pulse wave propagation time between a carotid artery and an arteriole or a capillary near the carotid artery, a pulse wave propagation time between the heart and the carotid artery and a pulse wave propagation time between the heart and the arteriole or the capillary can be obtained. Therefore, blood pressure at an artery can be estimated, and together with estimated blood pressure of an arteriole or a capillary, circulatory dynamics can be measured more accurately.

In the blood pressure status measuring apparatus according to the present invention, preferably, the biological sensor is heart sound acquiring means for acquiring a heart sound signal, and the pulse wave propagation time acquiring means acquires a pulse wave propagation time based on the photoplethysmographic signal of the arteriole or the capillary acquired by the photoplethysmographic sensor and the heart sound signal acquired by the heart sound acquiring means.

In this case, a pulse wave propagation time between the heart and an arteriole or a capillary can be obtained on the basis of a photoplethysmographic signal of the arteriole or the capillary and an electrocardiographic signal.

The blood pressure status measuring apparatus according to the present invention, preferably, further includes heart sound acquiring means for acquiring a heart sound signal. It is preferable that the pulse wave propagation time acquiring means acquires, in addition to the pulse wave propagation time based on the photoplethysmographic signal of the arteriole or the capillary acquired by the photoplethysmographic sensor and the pulse wave signal of the artery acquired by the pulse wave detecting means, a pulse wave propagation time based on the photoplethysmographic signal of the arteriole or the capillary acquired by the photoplethysmographic sensor and the heart sound signal acquired by the heart sound acquiring means and a pulse wave propagation time based on the pulse wave signal of the artery acquired by the pulse wave detecting means and the heart sound signal acquired by the heart sound acquiring means.

In this case, heart sound (in particular, the first sound) is measured at the same time as a reference. Thus, in addition to a pulse wave propagation time between a carotid artery and an arteriole or a capillary near the carotid artery, a pulse wave propagation time between the heart and the carotid artery and a pulse wave propagation time between the heart and the arteriole or the capillary can be obtained. Therefore, blood pressure at an artery can be estimated, and together with estimated blood pressure of an arteriole or a capillary, circulatory dynamics can be measured more accurately.

In the blood pressure status measuring apparatus according to the present invention, preferably, the photoplethysmographic sensor acquires a photoplethysmographic signal of an arteriole or a capillary near a carotid artery.

In this case, a pulse wave propagation time between a carotid artery and an arteriole or a capillary near the carotid artery is measured, and a temporal change of the pulse wave propagation time is measured. Therefore, a difference in blood pressure between the artery and the arteriole or the capillary can be measured, and circulatory dynamics can be measured. When a pulse wave propagation time is measured at an arteriole or a capillary near a carotid artery (a wide artery), it takes a time corresponding to the length of the arteriole or the capillary branching off from the carotid artery for arrival of a pulse wave. Therefore, the pulse wave propagation time is larger than a value measured at the carotid artery. Furthermore, blood pressure and circulatory dynamics of an arteriole or a capillary near, in particular, a carotid artery among arteries that can be measured relates to cardiovascular diseases, in particular, stroke. Therefore, estimation of blood pressure of the arteriole or the capillary near the carotid artery and observation of circulatory dynamics may be used for estimation of the risk of cardiovascular diseases.

The blood pressure status measuring apparatus according to the present invention, preferably, further includes posture detecting means for detecting posture of a user at a time when a pulse wave propagation time is acquired by the pulse wave propagation time acquiring means. It is preferable that the change acquiring means acquires a temporal change of the acquired pulse wave propagation time after measurement is started, in accordance with the posture detected by the posture detecting means.

A change (the amount of change and the rate of change) of the pulse wave propagation time of an arteriole or a capillary after measurement is started is affected by posture. However, in this case, posture of a user is detected, and a temporal change of the acquired pulse wave propagation time after measurement is started is acquired in accordance with the detected posture (that is, taking into consideration the posture). Therefore, a temporal change of a pulse wave propagation time (blood pressure) can be measured in a stable manner without being affected by a change of posture.

In the blood pressure status measuring apparatus according to the present invention, preferably, the change acquiring means sets a reference posture from among detected postures, and obtains a temporal change of the pulse wave propagation time after measurement is started, on the basis of time-series data of the pulse wave propagation time of the reference posture.

As described above, a change (the amount of change and the rate of change) of a pulse wave propagation time of an arteriole or a capillary after measurement is started is affected by posture. However, in this case, a reference posture is set from among detected postures, and a temporal change of the pulse wave propagation time after measurement is started is obtained on the basis of time-series data of the pulse wave propagation time of the reference posture. Therefore, a temporal change of a pulse wave propagation time (blood pressure etc.) can be measured in a stable manner without being affected by a change of posture.

In the blood pressure status measuring apparatus according to the present invention, preferably, the change acquiring means sets a reference posture from among detected postures, corrects, in accordance with the reference posture, time-series data of a pulse wave propagation time classified into a posture different from the reference posture, and obtains a temporal change of the pulse wave propagation time after measurement is started, on the basis of the time-series data of the pulse wave propagation time of the reference posture and the corrected time-series data of the pulse wave propagation time.

As described above, a change (the amount of change and the rate of change) of a pulse wave propagation time of an arteriole or a capillary after measurement is affected by posture. However, in this case, a reference posture is set from among detected postures, time-series data of a pulse wave propagation time classified into a posture different from the reference posture is corrected in accordance with the reference posture, and a temporal change of the pulse wave propagation time after measurement is started is obtained on the basis of the time-series data of the pulse wave propagation time of the reference posture and the corrected time-series data of the pulse wave propagation time. Therefore, a temporal change of a pulse wave propagation time (blood pressure etc.) can be measured in a stable manner without being affected by a change of posture.

In the blood pressure status measuring apparatus according to the present invention, preferably, the change acquiring means obtains, for each of detected postures, a temporal change of the pulse wave propagation time after measurement is started, and the measuring means measures circulatory dynamics including a blood pressure status on the basis of temporal changes of pulse wave propagation times after measurement is started for the individual postures.

In this case, by measuring temporal changes of pulse wave propagation times after measurement is started for multiple postures, for example, a difference in blood pressure and the like of an arteriole or a capillary vertically below an artery from which the arteriole or the capillary branches off and an arteriole or a capillary vertically above the artery from which the arteriole or the capillary branches off can be measured, and circulatory dynamics can be observed more accurately.

The blood pressure status measuring apparatus according to the present invention, preferably, further includes pressure detecting means for detecting pressure of the photoplethysmographic sensor. It is preferable that the measuring means changes a conversion expression to be used to calculate blood pressure of an arteriole or a capillary on the basis of a pulse wave propagation time, in accordance with the pressure detected by the pressure detecting means.

Time to obtain a stable pulse wave propagation time and a value of the stable pulse wave propagation time vary according to the pressure of a photoplethysmographic sensor. That is, a change of a pulse wave propagation time of an arteriole or a capillary after measurement is started is affected by the pressure of the photoplethysmographic sensor. However, in this case, the pressure of the photoplethysmographic sensor is measured, and a constant of a conversion expression for a pulse wave propagation time and blood pressure of an arteriole or a capillary varies according to the pressure. Therefore, the accuracy of estimation of blood pressure of the arteriole or the capillary and accuracy of evaluation of circulatory dynamics can be improved.

The blood pressure status measuring apparatus according to the present invention, preferably, further includes a pressure adjustment mechanism for adjusting the pressure to a specific value in accordance with the pressure detected by the pressure detecting means.

In this case, with provision of a mechanism for adjusting pressure according to the measured pressure, the pressure can be maintained at an optimal value. Therefore, the accuracy of estimation of blood pressure and the like can be improved.

The blood pressure status measuring apparatus according to the present invention, preferably, further includes input means for receiving an operation for inputting a value of a height or a sitting height of a user. It is preferable that the measuring means obtains an arterial length between an aortic valve and a carotid artery on the basis of the value of the height or the sitting height received by the input means, and corrects a blood pressure value of an arteriole or a capillary on the basis of the arterial length.

Pulse wave propagation velocity is directly correlated with blood pressure. Therefore, if the length of an artery is known, by converting a pulse wave propagation time into pulse wave propagation velocity, the accuracy of estimation of blood pressure can be improved. In this case, the arterial length between an aortic valve and a neck region (carotid artery) is obtained on the basis of the received value of the height or the sitting height of a user, and the value of blood pressure of the arteriole or the capillary is corrected in accordance with the arterial length. Therefore, the accuracy of estimation of blood pressure and the like can be improved.

According to the present invention, circulatory dynamics including the blood pressure status of arterioles or capillaries can be measured more simply and more accurately.

### BRIEF DESCRIPTION OF THE DRAWINGS

FIG. 1 is a block diagram illustrating a configuration of a blood pressure status measuring apparatus using a pulse wave propagation time measuring device according to an embodiment.

FIG. 2 is a perspective view illustrating the external appearance of a blood pressure status measuring apparatus of a neck band type using a pulse wave propagation time measuring device according to an embodiment.

FIG. 3 is a diagram illustrating an example of a temporal change of a pulse wave propagation time (from the heart to an arteriole or a capillary) based on an electrocardiogram and photoplethysmographic pulse waves of an arteriole or a capillary in a neck region.

FIG. 4 is a diagram illustrating a difference between pulse wave propagation times (from the heart to an arteriole or a capillary) based on electrocardiograms and photoplethysmographic pulse waves of an arteriole or a capillary in a left lateral recumbent position and in a right lateral recumbent position in the case where a photoplethysmographic sensor is arranged on the left lateral side of the neck region.

FIG. 5 is a flowchart (page 1) illustrating a processing procedure of a blood pressure status measuring process by a blood pressure status measuring apparatus according to an embodiment.

FIG. 6 is a flowchart (page 2) illustrating a processing procedure of a blood pressure status measuring process by a blood pressure status measuring apparatus according to an embodiment.

FIG. 7 is a diagram for explaining the relationship of blood vessels (an artery, an arteriole, and a capillary), blood pressure, and pulse wave propagation times.

### DESCRIPTION OF THE PREFERRED EMBODIMENTS

Hereinafter, preferred embodiments of the present invention will be described in detail below with reference to drawings. In the drawings, the same or corresponding parts will be referred to with the same signs. Furthermore, the same signs will be assigned to the same elements, and redundant explanation will be omitted.

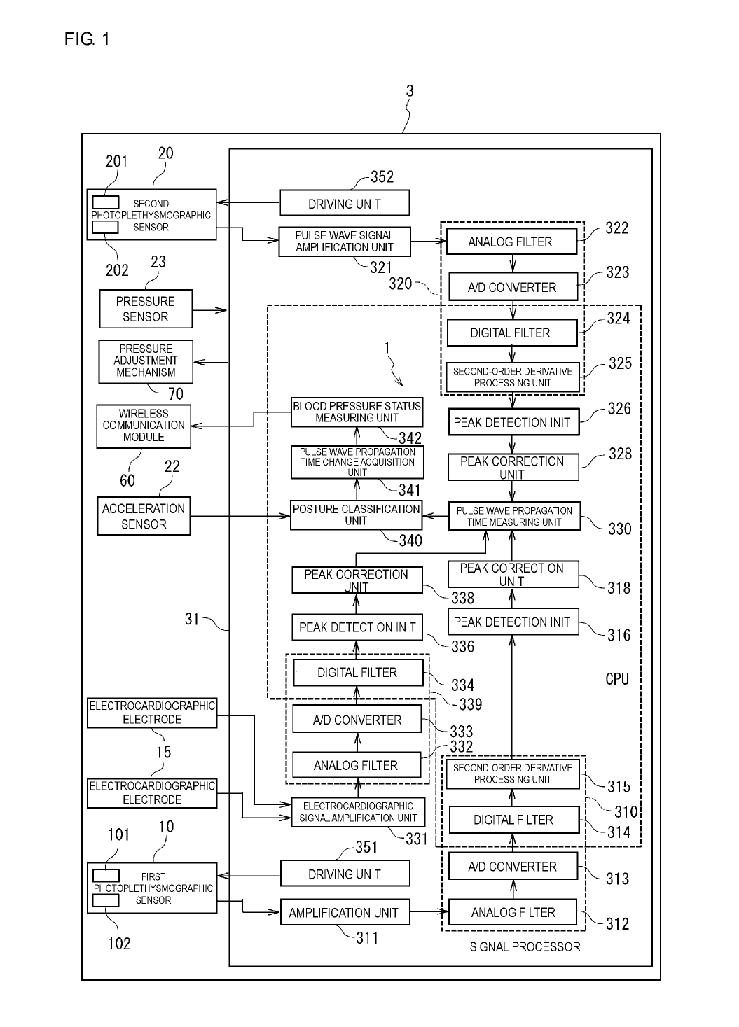

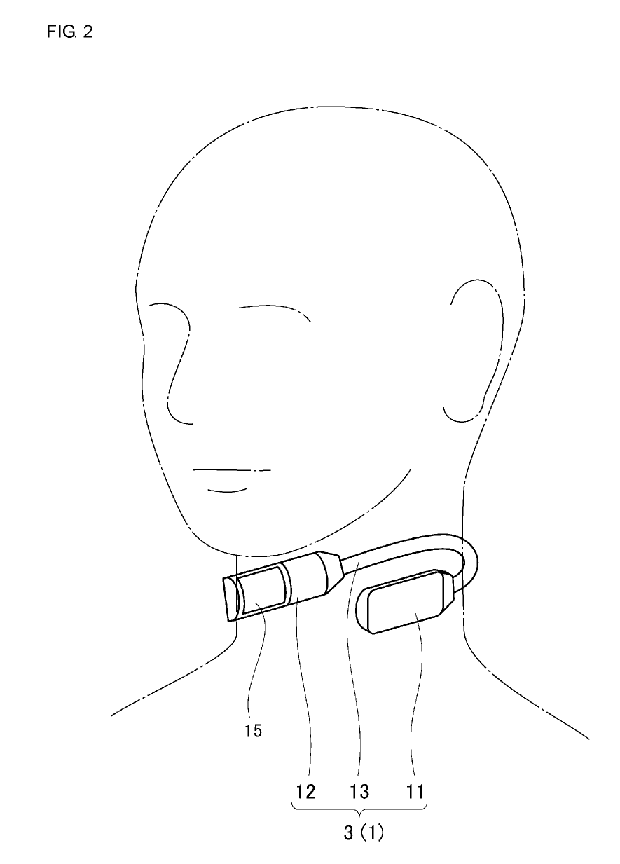

First, a configuration of a blood pressure status measuring apparatus 3 according to an embodiment will be explained with reference to FIGS. 1 and 2. The blood pressure status measuring apparatus 3 includes a pulse wave propagation time measuring device 1. FIG. 1 is a block diagram illustrating a configuration of the blood pressure status measuring apparatus 3 including the pulse wave propagation time measuring device 1. FIG. 2 is a perspective view illustrating the external appearance of the blood pressure status measuring apparatus 3 of a neck band type including the pulse wave propagation time measuring device 1.

The blood pressure status measuring apparatus 3 includes the pulse wave propagation time measuring device 1 that detects a first photoplethysmographic signal, a second photoplethysmographic signal, and an electrocardiographic signal and measures a pulse wave propagation time based on a time difference between the peak of the detected first photoplethysmographic signal, the peak of the detected second photoplethysmographic signal, and the peak of an R wave of the detected electrocardiographic signal. The blood pressure status measuring apparatus 3 estimates a change in blood pressure of a user on the basis of time-series data of calculated pulse wave propagation times. In particular, the blood pressure status measuring apparatus 3 has a function of estimating a change in blood pressure on the basis of time-series data (temporal change) of pulse wave propagation times and measuring, more simply and more accurately, circulatory dynamics including the blood pressure status of an arteriole or a capillary (arteriole or the like).

Thus, the blood pressure status measuring apparatus 3 mainly includes a first photoplethysmographic sensor 10 for detecting a first photoplethysmographic signal, a second photoplethysmographic sensor 20 for detecting a second photoplethysmographic signal, a pair of electrocardiographic electrodes 15 for detecting an electrocardiographic signal, an acceleration sensor 22 for detecting posture of a user, a pressure sensor 23 for detecting pressure of the first and second photoplethysmographic sensors 10 and 20, and a signal processor 31 that measures a pulse wave propagation time and the like from the detected first photoplethysmographic signal, second photoplethysmographic signal, and electrocardiographic signal and estimates circulatory dynamics including a change in blood pressure on the basis of a temporal change of pulse wave propagation times.

In this embodiment, as illustrated in FIG. 2, the blood pressure status measuring apparatus 3 is of a neck band type. For example, as illustrated in FIG. 2, the blood pressure status measuring apparatus 3 is worn on a neck region (nape of the neck) to obtain time-series data of pulse wave propagation times and estimate a change in blood pressure. The blood pressure status measuring apparatus 3 includes a neck band 13 of substantially a U-shape (or a C-shape) elastically worn such that the neck region of a user is held from the back side of the neck region and a pair of sensor units 11 and 12 that are arranged across the neck band 13 to be in contact with both sides of the neck region of the user.

The neck band 13 is wearable along the circumferential direction of the neck region of a user. That is, as illustrated in FIG. 2, the neck band 13 is worn along the back of the neck region of a user from one lateral side to the other lateral side of the neck region of the user. More specifically, the neck band 13 includes, for example, a band-shape plate spring and a rubber coat covering around the plate spring. Thus, the neck band 13 is energized to contract inwards. When a user is wearing the neck band 13, the neck band 13 (the sensor units 11 and 12) is maintained in contact with the neck region of the user.

It is desirable to use a rubber coat having biocompatibility. Furthermore, instead of a rubber coat, for example, a coat made of plastics may be used. Wiring cables for electrically connecting both the sensor units 11 and 12 are also arranged inside the rubber coat. To reduce noise, it is desirable that these cables are coaxial.

The sensor units 11 and 12 include respective electrocardiographic electrodes 15 (only one of which is shown in FIG. 2). The electrocardiographic electrodes 15 may be formed, for example, of silver/silver chloride, conductive gel, conductive rubber, conductive plastics, metal (desirably, high resistance to corrosion and unlikely to cause metal allergy, such as stainless or Au), conductive fabrics, capacitive coupling electrodes in which the surface of metal is coated with an insulating layer, or the like may be used. Exemplary conductive fabric are a woven fabric, a knitted fabric, a non-woven fabric made of conductive yarn having conductivity. An exemplary conductive yarns include resin yarns whose surface is Ag plated, carbon nanotube coated, or coated with a conductive polymer such as PEDOT. Furthermore, a conductive polymer yarn may be used. In this embodiment, conductive fabrics formed in a rectangular plan shape are used as the electrocardiographic electrodes 15. Each of the pair of electrocardiographic electrodes 15 and 15 is connected to the signal processor 31, and outputs an electrocardiographic signal to the signal processor 31.

Inside the sensor unit 11 (more generally, a surface that is in contact with the neck region), the first and second photoplethysmographic sensors 10 and 20 are arranged near the electrocardiographic electrode 15 and the acceleration sensor 22 (details of which will be described below).

The first photoplethysmographic sensor 10 is a sensor that optically detects a first photoplethysmographic signal by using light absorption characteristics of bloodstream hemoglobin. Thus, the first photoplethysmographic sensor 10 includes a first light emitting element 101 and a first light receiving element 102.

Similarly, the second photoplethysmographic sensor 20 is a sensor that optically detects a second photoplethysmographic signal by using light absorption characteristics of bloodstream hemoglobin. Thus, the second photoplethysmographic sensor 20 includes a second light emitting element 201 and a second light receiving element 202.

The first light emitting element 101 preferably emits light in accordance with a pulse-shape driving signal output from a driving unit 351 of the signal processor 31. As the first light emitting element 101, for example, an LED, a VCSEL (Vertical Cavity Surface Emitting LASER), a resonator-type LED, or the like may be used. The driving unit 351 generates and outputs a pulse-shape driving signal for driving the first light emitting element 101.

The first light receiving element 102 outputs a detection signal corresponding to the intensity of light applied from the first light emitting element 101 and incident by scatter reflection, for example, on skin. The first light receiving element 102 may be, for example, a photodiode, a phototransistor, or the like is preferably used. In this embodiment, a photodiode is used. The first light receiving element 102 is connected to the signal processor 31, and a detection signal (first photoplethysmographic signal) obtained by the first light receiving element 102 is output to the signal processor 31.

Similarly, the second light emitting element 201 emits light in accordance with a pulse-shape driving signal output from a driving unit 352 of the signal processor 31. As the second light emitting element 201, for example, an LED, a VCSEL, a resonator-type LED, or the like may be used. The driving unit 352 generates and outputs a pulse-shape driving signal for driving the second light emitting element 201.

The second light receiving element 202 outputs a detection signal corresponding to the intensity of light applied from the second light emitting element 201 and incident by scatter reflection, for example, on skin. As the second light receiving element 202, for example, a photodiode, a phototransistor, or the like is preferably used. In this embodiment, as the second light receiving element 202, a photodiode is used. The second light receiving element 202 is connected to the signal processor 31, and a detection signal (second photoplethysmographic signal) obtained by the second light receiving element 202 is output to the signal processor 31.

It is desirable that the first light emitting element 101 outputs near-infrared light with a wavelength of 800 to 1000 nm. In this embodiment, a first light emitting element that outputs near-infrared light with a wavelength of 850 nm is used. In contrast, it is desirable that the second light emitting element 201 outputs blue to yellow-green light with a wavelength of 450 to 580 nm. In this embodiment, a second light emitting element that outputs green light with a wavelength of 525 nm is used. Furthermore, the distance between the second light emitting element 201 and the second light receiving element 202 is set to be shorter than the distance between the first light emitting element 101 and the first light receiving element 102.

Blue to yellow-green light is highly absorbed by a living body. Therefore, a large photoplethysmographic signal can be obtained. However, the photoplethysmographic signal attenuates fast inside the living body, and a long optical path length cannot be obtained. In contrast, near-infrared light is not highly absorbed by a living body. Therefore, a smaller photoplethysmographic signal, but a longer optical path length, can be obtained. Thus, although measurement for a first photoplethysmographic signal and a second photoplethysmographic signal may be performed by using light having the same wavelength, it is desirable that the first photoplethysmographic sensor 10 having a long optical path length uses near-infrared light and the second photoplethysmographic sensor 20 having a short optical path length uses blue to yellow-green light.

Exemplary methods for isolating a first photoplethysmographic signal from a second photoplethysmographic signal are: one based on time division (a method for emitting detection beams in a pulse shape and shifting the light emitting timing), one based on wavelength division (a method for arranging a wavelength filter corresponding to each wavelength in front of a light receiving element), one based on space division (a method for arranging detection beams away from each other so as not to interfere with each other). Other methods can also be used.

With the configuration described above, the second photoplethysmographic sensor 20 having a short optical path length detects a second photoplethysmographic signal corresponding to the blood flow of an arteriole or a capillary at a position relatively close to the outermost layer of skin (that is, a shallow position). In contrast, the first photoplethysmographic sensor 10 having a long optical path length detects a first photoplethysmographic signal corresponding to the blood flow of an artery, which is wider than an arteriole or a capillary, at a position relatively far from the outermost layer of skin (that is, a deep position). Such a sensor is a form of a biological sensor.

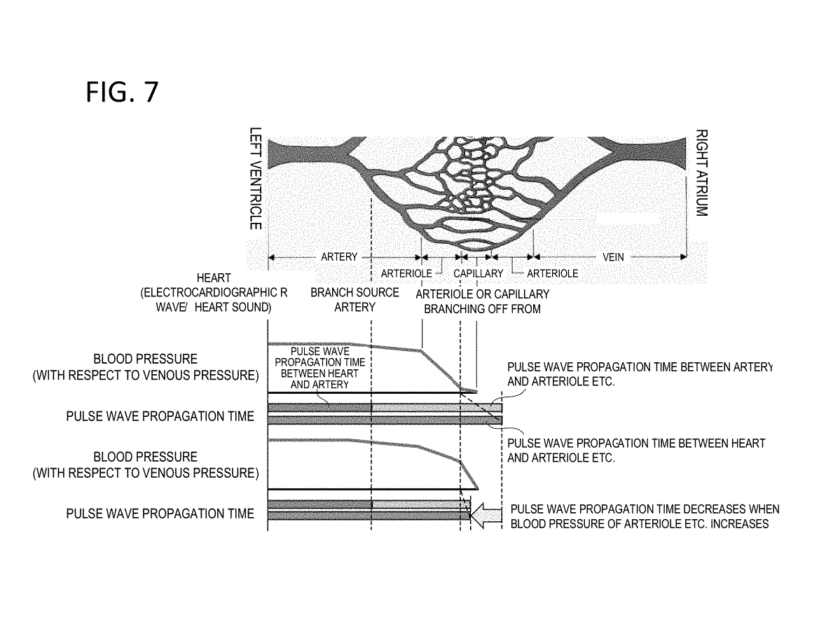

More particularly, for the second photoplethysmographic sensor 20 having a short optical path length, a photoplethysmographic signal contains little information of a wide carotid artery but contains much information of an arteriole or a capillary. In contrast, for the first photoplethysmographic sensor 10 having a long optical path length, a photoplethysmographic signal contains both information of a wide carotid artery and information of an arteriole and a capillary. Normally, however, since a signal regarding a wide carotid artery is larger than a signal regarding an arteriole or a capillary, the information regarding the wide carotid artery is superior to the information regarding the arteriole or the capillary for the long optical path length. A pulse wave sent from the heart reaches a carotid artery, branches out, and then reaches an arteriole or a capillary. Therefore, there is a time difference between arrival at the carotid artery and arrival at the arteriole or the capillary. Thus, by measuring photoplethysmographic signals at the carotid artery and the arteriole or the capillary near the carotid artery, pulse wave propagation times can be measured at substantially the same part. An arteriole is a narrow artery with a diameter of, for example, 10 to 100 μm and is a blood vessel existing at a place between the artery and a capillary. Furthermore, a capillary is a narrow blood vessel that connects an artery to a vein and has a diameter of, for example, 5 to 10 μm (see FIG. 7).

The first photoplethysmographic sensor 10 and the second photoplethysmographic sensor 20 are arranged so as to be in contact with the outermost layer of skin above a carotid artery when they are worn. The first photoplethysmographic sensor 10 detects a first photoplethysmographic signal corresponding to the blood flow of the carotid artery. In contrast, the second photoplethysmographic sensor 20 detects a photoplethysmographic signal corresponding to the blood flow of an arteriole or a capillary near the carotid artery branching off from the carotid artery. It is desirable that the first photoplethysmographic sensor 10 and the second photoplethysmographic sensor 20 are arranged so as to be in contact with a left lateral side of the neck region (immediately above a carotid artery and near the part immediately above the carotid artery (for example, within 10 cm)). In this case, for example, in a left lateral recumbent position, a right lateral recumbent position, and a supine position, the height of the left ventricle, which is a reference for blood pressure, is substantially the same as the height of the first photoplethysmographic sensor 10 and the second photoplethysmographic sensor 20. Therefore, a change in blood pressure can be measured in a stable manner, regardless of the type of lying position. The left ventricle is located to the slightly left of the center of the chest. Therefore, by arranging the first photoplethysmographic sensor 10 and the second photoplethysmographic sensor 20 on the left lateral side of the neck region, a deviation in the horizontal direction between the left ventricle and each of the first photoplethysmographic sensor 10 and the second photoplethysmographic sensor 20 is reduced. Furthermore, in a supine position, the left ventricle is located closer to the chest than the back. However, in a supine position without using a pillow, the neck region is positioned lower than the chest. By arranging the first photoplethysmographic sensor 10 and the second photoplethysmographic sensor 20 on a left lateral side of the neck region when a pillow is used, although depending on the height of the pillow, a deviation in the height with respect to the left ventricle in a supine position can be reduced.

Furthermore, the acceleration sensor 22 that detects the posture of a user (neck region) at a time when obtaining a pulse wave propagation time is attached to the sensor unit 11. That is, the acceleration sensor 22 functions as posture detecting means. The acceleration sensor 22 is preferably a 3-axis acceleration sensor that detects a direction to which gravitational acceleration G is applied (that is, a vertical direction). The acceleration sensor 22 is able to determine, based on the detection signal, for example, whether the user is standing or lying.

More specifically, the positional relationship of the acceleration sensor 22 relative to the body of a user is calibrated in advance. For example, posture of the user can be determined by performing coordinates conversion such that a direction to which gravitational acceleration is applied when the user is standing is defined as a downward direction (vertical direction) relative to output of the acceleration sensor 22. The acceleration sensor 22 is also connected to the signal processor 31, and outputs a detection signal (3-axis acceleration data) to the signal processor 31. In place of the acceleration sensor 22, for example, a gyro sensor or the like may be used.

The first photoplethysmographic sensor 10, the second photoplethysmographic sensor 20, and the acceleration sensor 22 are preferably arranged adjacent to one another. For use (measurement), the first photoplethysmographic sensor 10, the second photoplethysmographic sensor 20, and the acceleration sensor 22 are worn on the neck region (neck) of a user. As described above, by wearing the first photoplethysmographic sensor 10, the second photoplethysmographic sensor 20, and the acceleration sensor 22 for determining posture on the same part (e.g., on the device of FIG. 2), correlation between posture termination and pulse wave propagation time can be increased. Furthermore, by wearing them on the neck region (or the trunk of the body) not on limbs or the like, intravascular pressure of the neck region (or the trunk of the body) that is presumed to be highly correlated with the risk of stroke, myocardial infarction, or the like, not intravascular pressure of the limbs, can be estimated. Furthermore, by wearing all the multiple sensors on the neck region (or the trunk of the body) not on separate parts, trouble in wearing can be reduced, and restriction in daily life can be reduced.

In the sensor unit 11, the pressure sensor 23 that detects pressure (stress) applied to skin of a user is attached near the first photoplethysmographic sensor 10 and the second photoplethysmographic sensor 20. The pressure sensor 23 functions as pressure detecting means. For example, a force sensor or a strain sensor such as a piezoelectric sensor, a strain gauge, or the like, or a sensor for detecting deformation of a piezoelectric film may be used as the pressure sensor 23. When pressure is low, a long time is required to obtain a stable pulse wave propagation time. Therefore, the time for determining that pulse wave propagation time has entered a stable state is changed according to the pressure (details will be described below).

A pressure adjustment mechanism 70 that adjusts pressure of the first photoplethysmographic sensor 10 and the second photoplethysmographic sensor 20 to a specific value according to the pressure detected by the pressure sensor 23 may further be added to the sensor unit 11. In this case, it is determined whether or not a measured pressure falls within an appropriate pressure range. In the case where the measured pressure does not fall within the appropriate pressure range, a pressure adjustment signal is output to the pressure adjustment mechanism 70. More specifically, for example, in the case where the detected pressure is low, a mechanism for causing the photoplethysmographic sensors 10 and 20 to protrude toward the neck region relative to the neck band 13 is added, a mechanism for reducing stretch of the neck band 13 is added, or an air bag is inflated by a pump so as to press the photoplethysmographic sensors 10 and 20 out to the neck region side, so that the pressure can be increased. By changing the area that is in contact with skin in accordance with the pressure (by increasing the contact area when the pressure is high), pain and occurrence of indentation in skin can be reduced. For example, with the use of a housing that is elastically deformed with little force, a structure in which the housing is deformed and the contact area thus increases in the case where the pressure is high, can be achieved.

Furthermore, a battery (not shown) that supplies electric power to the first photoplethysmographic sensor 10, the second photoplethysmographic sensor 20, the signal processor 31, a wireless communication module 60, and the like are accommodated within the sensor unit 11. The signal processor 31 and the wireless communication module 60 that transmits biological information such as a blood pressure status (circulatory dynamics), a measured pulse wave propagation time, an electrocardiographic signal, and a photoplethysmographic signal to an external apparatus are preferably accommodated within the sensor unit 12.

As described above, each of the electrocardiographic electrodes 15 and 15, the first photoplethysmographic sensor 10, and the second photoplethysmographic sensor 20 is connected to the signal processor 31, and the detected electrocardiographic signal, first photoplethysmographic signal, and second photoplethysmographic signal are input to the signal processor 31. Hereinafter, a first photoplethysmographic signal and a second photoplethysmographic signal may be collectively and simply referred to as photoplethysmographic signals. Furthermore, the acceleration sensor 22 and the pressure sensor 23 are also connected to the signal processor 31, and the detected 3-axis acceleration signal and pressure signal are input to the signal processor 31.

The signal processor 31 preferably measures a pulse wave propagation time and the like on the basis of each of a time difference between a rising point (peak) of a detected first photoplethysmographic signal (or an acceleration pulse wave signal) and a rising point (peak) of a second photoplethysmographic signal (or an acceleration pulse wave signal), a time difference between the rising point (peak) of the first photoplethysmographic signal (or the acceleration pulse wave signal) and an R wave of an electrocardiographic signal, and a time difference between the rising point (peak) of the second photoplethysmographic signal (or the acceleration pulse wave signal) and the R wave of the electrocardiographic signal. Then, the signal processor 31 measures and estimates a change in blood pressure of a user, circulatory dynamics, and the like on the basis of time-series data (a temporal change, that is a change overtime) of the measured pulse wave propagation times. Furthermore, the signal processor 31 processes the input photoplethysmographic signals, and calculates the pulse rate, the pulse interval, and the like. Furthermore, the signal processor 31 processes the input electrocardiographic signals, and calculates the heart rate, the heartbeat interval, and the like.

Therefore, the signal processor 31 includes amplification units 311, 321, and 331, a first signal processing unit 310, a second signal processing unit 320, a third signal processing unit 339, peak detection units 316, 326, and 336, peak correction units 318, 328, and 338, a pulse wave propagation time measuring unit 330, a posture classification unit 340, a pulse wave propagation time change acquisition unit 341, and a blood pressure status measuring unit 342. Furthermore, the first signal processing unit 310 includes an analog filter 312, an A/D converter 313, a digital filter 314, and a second-order derivative processing unit 315. In contrast, the second signal processing unit 320 includes an analog filter 322, an A/D converter 323, a digital filter 324, and a second-order derivative processing unit 325. Furthermore, the third signal processing unit 339 includes an analog filter 332, an A/D converter 333, and a digital filter 334.

Among the units described above, the digital filters 314, 324, and 334, the second-order derivative processing units 315 and 325, the peak detection units 316, 326, and 336, the peak correction units 318, 328, and 338, the pulse wave propagation time measuring unit 330, the posture classification unit 340, the pulse wave propagation time change acquisition unit 341, and the blood pressure status measuring unit 342 each include a CPU that performs arithmetic processing, a ROM that stores a program and data for causing the CPU to perform each process, a RAM that temporarily stores various data such as arithmetic results, and the like. That is, when a program stored in the ROM is executed by the CPU, a function of each unit described above is implemented.

The amplification unit 311 preferably includes, for example, an amplifier such as an operational amplifier, and amplifies a first photoplethysmographic signal detected by the first photoplethysmographic sensor 10. The first photoplethysmographic signal amplified by the amplification unit 311 is output to the first signal processing unit 310. Similarly, the amplification unit 321 preferably includes, for example, an amplifier such as an operational amplifier, and amplifies a second photoplethysmographic signal detected by the second photoplethysmographic sensor 20. The second photoplethysmographic signal amplified by the amplification unit 321 is output to the second signal processing unit 320. Furthermore, the amplification unit 331 preferably includes, for example, an amplifier such as an operational amplifier, and amplifies electrocardiographic signals detected by the electrocardiographic electrodes 15 and 15. The electrocardiographic signals amplified by the amplification unit 331 are output to the third signal processing unit 339.

As described above, the first signal processing unit 310 includes the analog filter 312, the A/D converter 313, the digital filter 314, and the second-order derivative processing unit 315, and extracts a pulsation component by performing filtering processing and second-order derivative processing on the first photoplethysmographic signal amplified by the amplification unit 311.

Furthermore, as described above, the second signal processing unit 320 preferably includes the analog filter 322, the A/D converter 323, the digital filter 324, and the second-order derivative processing unit 325, and extracts a pulsation component by performing filtering processing and second-order derivative processing on a second photoplethysmographic signal amplified by the amplification unit 321.

As described above, the third signal processing unit 339 preferably includes the analog filter 332, the A/D converter 333, and the digital filter 334, and extracts a heartbeat component by performing filtering processing on an electrocardiographic signal amplified by the amplification unit 331.

The analog filters 312, 322, and 332 and the digital filters 314, 324, and 334 eliminate components (noise) other than frequencies characterizing photoplethysmographic signals and an electrocardiographic signal, and preferably perform filtering to improve S/N. More particularly, for a photoplethysmographic signal, a frequency component of about 0.1 to several tens of Hz is dominant. For an electrocardiographic signal, in general, a frequency component of 0.1 to 200 Hz is dominant. Therefore, S/N is improved by performing filtering processing using the analog filters 312, 322, and 332 and the digital filters 314, 324, and 334 such as low pass filters and band pass filters and allowing only signals in the above-mentioned frequency ranges to selectively pass through the filters.

For the case where only extraction of pulsation components is required, in order to improve noise tolerance, a pass frequency range of the filters is further reduced so that components other than pulsation components can be blocked. Furthermore, some or all of the analog filters 312, 322, and 332 and the digital filters 314, 324, and 334 can be omitted. Only one of the analog filters 312, 322, and 332 and the digital filters 314, 324, and 334 may be provided. A first photoplethysmographic signal that has been subjected to filtering processing by the analog filter 312 and the digital filter 314 is output to the second-order derivative processing unit 315. Similarly, a photoplethysmographic signal that has been subjected to filtering processing by the analog filter 322 and the digital filter 324 is output to the second-order derivative processing unit 325. Furthermore, an electrocardiographic signal that has been subjected to filtering processing by the analog filter 332 and the digital filter 334 is output to the peak detection unit 336.

The second-order derivative processing unit 315 preferably performs second-order derivative of a first photoplethysmographic signal to obtain a first second-order derivative pulse wave (acceleration pulse wave) signal. The obtained first acceleration pulse wave signal is output to the peak detection unit 316. A change of the photoplethysmographic peak is not clear and may be difficult to detect. Therefore, it is desirable to perform conversion into an acceleration pulse wave so that peak detection can be performed. However, the second-order derivative processing unit 315 can be omitted. A configuration not including the second-order derivative processing unit 315 is also possible.

Similarly, the second-order derivative processing unit 325 preferably performs second-order derivative of a photoplethysmographic signal to obtain a second-order derivative pulse wave (acceleration pulse wave) signal. The obtained acceleration pulse wave signal is output to the peak detection unit 326. The second-order derivative processing unit 325 can be omitted. A configuration not including the second-order derivative processing unit 325 is also possible.

The peak detection unit 316 detects the peak of a first photoplethysmographic signal (acceleration pulse wave) that has been subjected to filtering processing by the first signal processing unit 310. In contrast, the peak detection unit 326 detects the peak of a second photoplethysmographic signal (acceleration pulse wave) that has been subjected to filtering processing by the second signal processing unit 320. Furthermore, the peak detection unit 336 detects the peak (R wave) of an electrocardiographic signal that has been subjected to signal processing by the third signal processing unit 339 (from which a pulsation component has been extracted). Each of the peak detection unit 316, the peak detection unit 326, and the peak detection unit 336 preferably performs peak detection within a normal range of pulse interval and heartbeat interval, and information of the peak time, peak amplitude, and the like of all the detected peaks is stored in the RAM or the like.

The peak correction unit 318 obtains a delay time of a first photoplethysmographic signal in the first signal processing unit 310 (the analog filter 312, the A/D converter 313, the digital filter 314, and the second-order derivative processing unit 315). The peak correction unit 318 corrects the peak of the first photoplethysmographic signal (acceleration pulse wave signal) detected by the peak detection unit 316, based on the obtained delay time of the first photoplethysmographic signal. Similarly, the peak correction unit 328 obtains a delay time of a second photoplethysmographic signal in the second signal processing unit 320 (the analog filter 322, the A/D converter 323, the digital filter 324, and the second-order derivative processing unit 325). The peak correction unit 328 corrects the peak of the second photoplethysmographic signal (acceleration pulse wave signal) detected by the peak detection unit 326, based on the obtained delay time of the second photoplethysmographic signal. Furthermore, the peak correction unit 338 obtains a delay time of an electrocardiographic signal in the third signal processing unit 339 (the analog filter 332, the A/D converter 333, and the digital filter 334). The peak correction unit 338 corrects the peak of the electrocardiographic signal detected by the peak detection unit 336, based on the obtained delay time of the electrocardiographic signal. Each of the corrected peak of the first photoplethysmographic signal (acceleration pulse wave), the corrected peak of the second photoplethysmographic signal (acceleration pulse wave), and the corrected peak of the electrocardiographic signal is output to the pulse wave propagation time measuring unit 330. In the case where the delay times of the first photoplethysmographic signal (acceleration pulse wave signal), the second photoplethysmographic signal (acceleration pulse wave signal), and the electrocardiographic signal may be regarded as being substantially the same, the peak correction units 318, 328, and 338 are not essentially provided, and an omitted configuration is also possible.

The pulse wave propagation time measuring unit 330 chronologically acquires pulse wave propagation times, based on each of the interval (time difference) between the peak of the first photoplethysmographic signal (acceleration pulse wave) corrected by the peak correction unit 318 and the peak of the second photoplethysmographic signal (acceleration pulse wave) corrected by the peak correction unit 328, the interval (time difference) between the peak of the first photoplethysmographic signal (acceleration pulse wave) corrected by the peak correction unit 318 and the peak of the electrocardiographic signal corrected by the peak correction unit 338, and the interval (time difference) between the peak of the second photoplethysmographic signal (acceleration pulse wave) corrected by the peak correction unit 328 and the peak of the electrocardiographic signal corrected by the peak correction unit 338. That is, as illustrated in FIG. 7, the pulse wave propagation time measuring unit 330 chronologically acquires pulse wave propagation time between the heart and a carotid artery, the pulse wave propagation time between the carotid artery and an arteriole or a capillary branching off from the carotid artery, and the pulse wave propagation time between the heart and the arteriole or the capillary branching off from the carotid artery. FIG. 7 is a diagram for explaining the relationship of blood vessels (an artery, an arteriole, and a capillary), blood pressure, and pulse wave propagation times. As illustrated in FIG. 7 (lower part), in the case where blood pressure of an arteriole or the like increases, the pulse wave propagation time between the heart and a carotid artery does not change, whereas the pulse wave propagation time between the carotid artery and the arteriole or the capillary branching off from the carotid artery and the pulse wave propagation time between the heart and the arteriole or the capillary branching off from the carotid artery decrease. The pulse wave propagation time measuring unit 330 functions as pulse wave propagation time acquiring means.

The pulse wave propagation time measuring unit 330 calculates, in addition to the pulse wave propagation time, for example, the heart rate, the heartbeat interval, the heartbeat interval change rate, and the like, based on an electrocardiographic signal. Similarly, the pulse wave propagation time measuring unit 330 calculates the pulse rate, the pulse interval, the pulse interval change range, and the like, based on a photoplethysmographic signal (acceleration pulse wave). The acquired time-series data of pulse wave propagation times are output to the posture classification unit 340.

The posture classification unit 340 determines (estimates) the posture of a user on the basis of a detection signal (3-axis acceleration data) of the acceleration sensor 22, and classifies time-series data of the above-described three pulse wave propagation times according to the determined posture. More specifically, the posture classification unit 340 classifies time-series data of pulse wave propagation times according to the posture of the user (patient) into at least a standing position, an inverted standing position, a supine position, a left lateral recumbent position, a right lateral recumbent position, and a prone position.

The pulse wave propagation time change acquisition unit 341 obtains a change of pulse wave propagation time from the beginning of measurement on the basis of each piece of time-series data of pulse wave propagation times classified by the posture classification unit 340 according to the posture. That is, the pulse wave propagation time change acquisition unit 341 functions as change acquiring means.

More specifically, the pulse wave propagation time change acquisition unit 341 first sets a reference posture (for example, a supine position) from among classified postures, and corrects, in accordance with the reference posture, time-series data of pulse wave propagation times classified into postures (for example, a standing position, an inversed standing position, a left lateral recumbent position, a right lateral recumbent position, and a prone position) different from the reference posture. Then, the pulse wave propagation time change acquisition unit 341 obtains a change of pulse wave propagation time on the basis of time-series data of the pulse wave propagation times for the reference posture and the corrected time-series data of the pulse wave propagation times (after correction).

At this time, the pulse wave propagation time change acquisition unit 341 sets a posture (for example, a supine position) for which time of the acquired time-series data of pulse wave propagation times is the longest as a reference posture. Then, the pulse wave propagation time change acquisition unit 341 corrects the time-series data of a pulse wave propagation time for each posture such that a correlation coefficient of an approximate curve obtained by approximating time-series data of pulse wave propagation times for each posture by a curve is large (preferably, maximum), and obtains a change of pulse wave propagation time on the basis of the corrected time-series data. As described above, by correcting the pulse wave propagation time for each posture such that the correlation coefficient of an approximate curve is large and estimating tendency of a change of pulse wave propagation time on the basis of the corrected time-series data, tendency of a change of pulse wave propagation time for a long time (tendency of a change in blood pressure) can be estimated without troublesome calibration even in the case where there is a change in posture. For example, a least squares method can be used as a method for calculating the approximate curve as mentioned above.

In place of the above-described method, pulse wave propagation times for individual postures may be arranged in chronological order and approximate curves for the individual pulse wave propagation times may be obtained. In this case, multiple approximate curves are calculated. However, an approximate curve having a large correlation coefficient is selected from among approximate curves for postures with a specific time ratio or more. Change data of pulse wave propagation time obtained by the pulse wave propagation time change acquisition unit 341 is output to the blood pressure status measuring unit 342.

The blood pressure status measuring unit 342 measures circulatory dynamics including the blood pressure status of an arteriole or a capillary on the basis of a temporal change from the beginning of measurement of a pulse wave propagation time (for example, a change from the beginning of measurement to acquisition of a stable pulse wave propagation time, that is, an initial value and a change with the lapse of time from the beginning). That is, the blood pressure status measuring unit 342 functions as measuring means. Circulatory dynamics represent the status of blood flowing in a circulatory system including blood vessels, the heart, and the like. Furthermore, circulation is formed by three elements: heart, blood vessels, and circulating blood volume.

First, the blood pressure status measuring unit 342 estimates a change in blood pressure on the basis of change data of each corrected pulse wave propagation time and the relationship (correlation expression) of a predetermined pulse wave propagation time and blood pressure of an arteriole or a capillary. For example, the blood pressure status measuring unit 342 estimates a change in blood pressure in accordance with a correlation expression (conversion expression) of a pulse wave propagation time and blood pressure for a reference posture (for example, a supine position) obtained in advance, so that a change in blood pressure can be estimated on the basis of a change in the corrected pulse wave propagation time. The correlation expression of a pulse wave propagation time and blood pressure may be obtained based on a posture different from a supine position or may be obtained collectively for multiple postures. It is desirable that, for measurement of circulatory dynamics including the status of blood pressure based on a temporal change of pulse wave propagation time, the correction expression (conversion expression) (or a constant of the correction expression (conversion expression)) is changed according to the pressure of the photoplethysmographic sensors 10 and 20.

The blood pressure status measuring unit 342 may perform, in advance, calibration for determining posture in a wearing state, that is, calibration of the relationship of an output signal (vertical direction) of the acceleration sensor 22 and posture of a user (for example, a standing position or a supine position), obtain a relational expression of a deviation of an angle (deviation angle) from the reference posture and the height from the heart to a pulse wave measurement part (that is, a part where the photoplethysmographic sensor 20 is worn (in this embodiment, a neck region)), and cause the obtained relational expression to be stored in a memory such as a RAM. For measurement (use) of a pulse wave propagation time, the blood pressure status measuring unit 342 may calculate a deviation of an angle (deviation angle) of the posture of the user detected by the acceleration sensor 22 and the reference posture on the basis of a result of the calibration performed in advance. For calculation of a blood pressure value based on the pulse wave propagation time, the blood pressure status measuring unit 342 may obtain the height from the heart to the pulse wave measurement part (the part where the photoplethysmographic sensor 20 is worn (neck region)) on the basis of the calculated deviation of the angle (deviation angle) and the above-mentioned relational expression stored in advance, and correct the blood pressure value in accordance with the height.

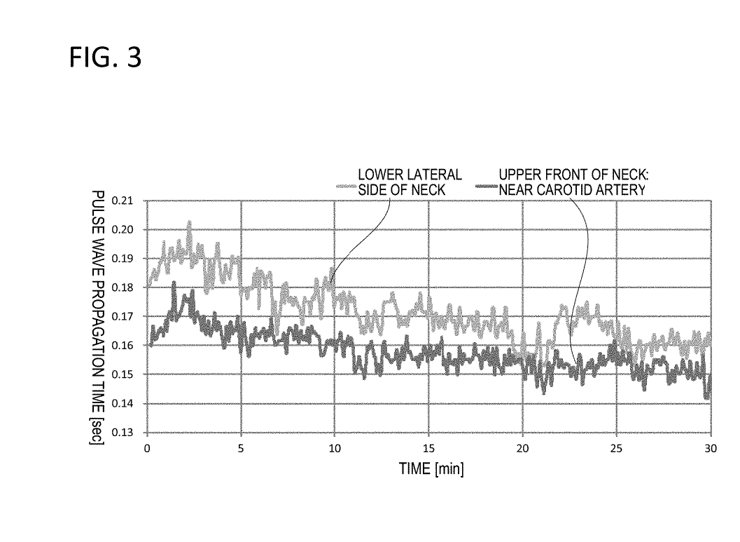

When a pulse wave is measured at an arteriole or a capillary near a carotid artery (a wide artery), it takes a time corresponding to the length of the arteriole or the capillary branching off from the carotid artery. Therefore, a pulse wave propagation time is larger than a value measured at the carotid artery. FIG. 3 illustrates an example of a temporal change of a pulse wave propagation time (a lower lateral side of the neck and an upper front of the neck) based on an electrocardiogram and photoplethysmographic pulse waves of an arteriole or a capillary in the neck region. As illustrated in FIG. 3, when pressure is applied, the pressure of the arteriole or capillary gradually increases, and the pulse wave propagation time decreases to approach a value at the carotid artery and then becomes stable. Furthermore, temporal changes of pulse wave propagation times are substantially the same even if pulse waves are measured at different positions, and circulatory dynamics of the arteriole or capillary are substantially the same between the upper front of the neck and the lower lateral side of the neck. The upper front of the neck is a part near the carotid artery, and the length of the arteriole or the capillary branching off from the carotid artery is short. Therefore, the value of the pulse wave propagation time is overall smaller than that for the lower lateral side of the neck.

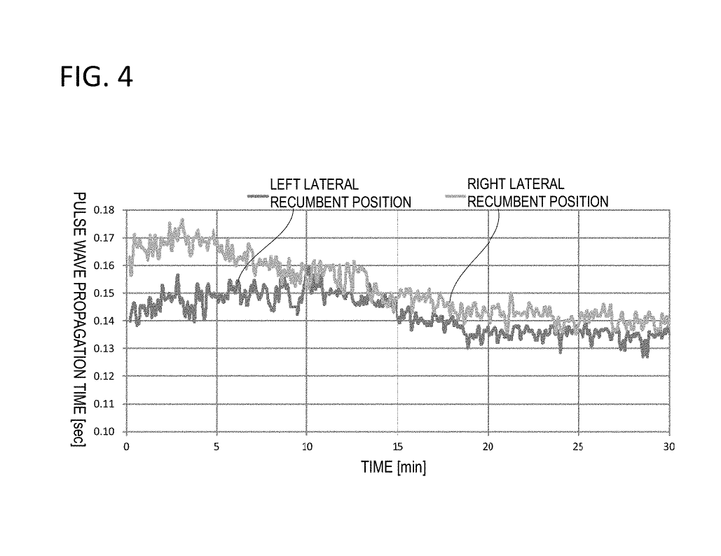

Furthermore, the temporal change (the amount of change and the rate of change) of a pulse wave propagation time varies according to the posture. A difference in temporal change of pulse wave propagation times (in a left lateral recumbent position and a right lateral recumbent position) based on electrocardiograms and photoplethysmographic pulse waves of an arteriole or a capillary in the left lateral recumbent position and the right lateral recumbent position in the case where the photoplethysmographic sensors 10 and 20 are arranged on the left lateral side of the neck region is illustrated in FIG. 4. The photoplethysmographic sensors 10 and 20 are arranged so as to be in contact with the left lateral side of the neck region, and the left ventricle and the pulse wave sensors are located at substantially the same height both in the left lateral recumbent position and the right lateral recumbent position. Therefore, when a sufficient time has passed, two pulse wave propagation times reach substantially the same value. In the case where measurement is performed in an upper part of the neck region (right lateral recumbent position), the initial pulse wave propagation time is large, and the amount of decrease is large. Therefore, the blood pressure of an arteriole at a vertically upper part of the body is lower than the blood pressure of an arteriole at a vertically lower part. When pressure is applied, the blood pressure of an arteriole or a capillary increases.

That is, by measuring a pulse wave propagation time at the beginning of measurement and a temporal change of the pulse wave propagation time, the blood pressure of an arteriole or a capillary can be estimated. In particular, the amount of change of the pulse wave propagation time in the right lateral recumbent position is important for estimation of blood pressure of an arteriole or a capillary. Furthermore, by performing measurement with multiple postures, a difference in the blood pressure of an arteriole or a capillary between a vertically lower part and a vertically upper part can be estimated, and circulatory dynamics can be estimated. By measuring the pulse wave propagation time between a carotid artery and an arteriole or capillary near the carotid artery and measuring a temporal change of the pulse wave propagation time, a difference in blood pressure between the carotid artery and the arteriole or capillary can be estimated, and circulatory dynamics can thus be estimated. Furthermore, it is desirable that the circulatory dynamics of an artery is estimated from a pulse wave propagation time based on an electrocardiographic signal and a pulse wave signal of the artery and that the circulatory dynamics of an arteriole or a capillary is estimated from a pulse wave propagation time based on a pulse wave signal of an artery and a pulse wave signal of the arteriole or capillary.

Furthermore, in particular, blood pressure and circulatory dynamics of an arteriole or a capillary near a carotid artery out of arteries that may be measured are related to circulatory diseases, in particular, stroke. Therefore, estimation of blood pressure and estimation of circulatory dynamics of an arteriole or a capillary near a carotid artery may be used for estimation of the risk of circulatory diseases.

Application to carotid arteries has been mainly explained above. Application to estimation of the risk of a diseases different from those described above is possible by applying the present invention to other arteries. For example, in the case where an artery is a dorsalis pedis artery or a posterior tibial artery, blood pressure and circulatory dynamics of lower extremity arteriole or capillary can be estimated, and these estimations may be used for evaluation for vascular disorders, evaluation for sensitivity to cold for (ASO) arteriosclerosis obliterans, PAD (peripheral arterial disease), diabetic patients, dialysis patients. In the case where an artery is a radial artery, blood pressure and circulatory dynamics for an arteriole or capillary can be estimated, and the estimation may be used for evaluation for cold sensitivity.

Furthermore, by measuring heart sound (in particular, the first sound) at the same time as a reference, instead of an electrocardiogram (for example, an R wave), in addition to a pulse wave propagation time between a carotid artery and an arteriole or a capillary near the carotid artery, a pulse wave propagation time between the heart and the carotid artery and a pulse wave propagation time between the heart and the arteriole or the capillary can be obtained. In this case, blood pressure at an artery is estimated in accordance with the pulse wave propagation time from the heart to the carotid artery. Furthermore, blood pressure of the arteriole or the capillary is estimated in accordance with the pulse wave propagation time between the carotid artery and the arteriole or the capillary near the carotid artery. Thus, the blood pressure of an artery can be estimated, and together with estimated blood pressure of an arteriole or a capillary, evaluation of circulatory dynamics can be performed more accurately.

The blood pressure status measuring unit 342 may perform classification into a dipper type, a non-dipper type, a riser type, and an extreme dipper type, according to an estimated blood pressure change. At a normal time, a dipper type in which blood pressure decreases during sleep is obtained. However, a hypertensive patient has hypertension during night time or blood pressure does not decrease during night time (riser type or non-dipper type), and the risk of stroke, myocardial infarction, or the like increases. Furthermore, in the case of a patient taking anti-hypersensitive drug, blood pressure becomes too low during sleep (extreme dipper type), and the risk of stroke, myocardial infarction, or the like may be increased. Therefore, by acquiring a change in blood pressure during sleep, determination between the riser type, the non-dipper type, and the extreme dipper type can be performed.

The estimated blood pressure status, circulatory dynamics, and blood pressure value, the calculated measurement data such as a pulse wave propagation time, a heart rate, a heartbeat interval, a pulse rate, a pulse interval, a photoplethysmographic pulse wave, an acceleration pulse wave, and a 3-axis acceleration are output to a memory such as a RAM, the wireless communication module 60, and the like. These measurement data may be stored in a memory so that they may be read along with daily change history or may be wirelessly transmitted in real time to an external apparatus such as a personal computer (PC) or a smartphone. Furthermore, these measurement data may be stored in a memory of the apparatus during measurement and the apparatus may be automatically connected to an external apparatus so that the data may be transmitted after measurement is completed.

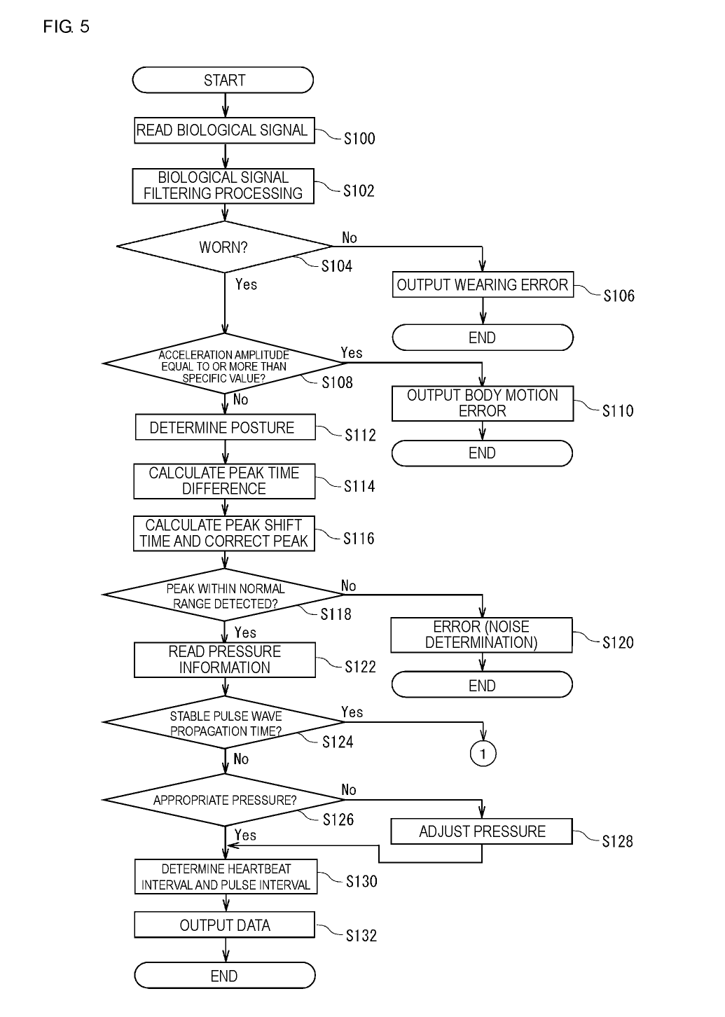

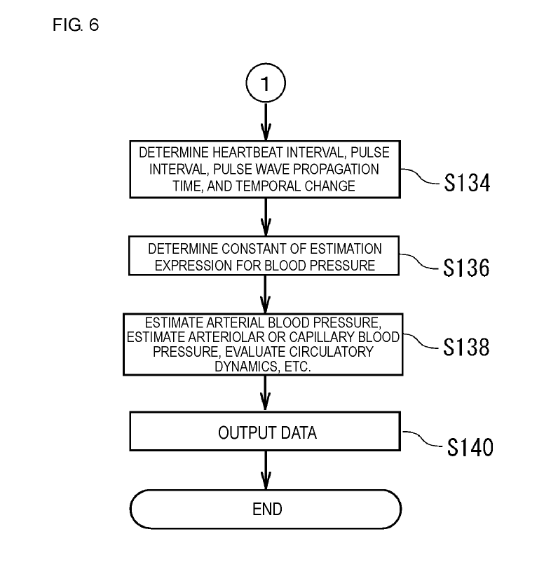

Next, an operation of the blood pressure status measuring apparatus 3 will be explained with reference to FIGS. 5 and 6. FIGS. 5 and 6 are flowcharts illustrating a processing procedure of a blood pressure status measuring process by the blood pressure status measuring apparatus 3. The process illustrated in FIGS. 5 and 6 is repeatedly performed at a specific timing mainly by the signal processor 31.

When the blood pressure status measuring apparatus 3 is worn on the neck region and the sensor units 11 and 12 containing respective electrocardiographic electrodes 15 and 15, the first photoplethysmographic sensor 10, and the second photoplethysmographic sensor 20) are made to be in contact with the neck region, electrocardiographic signals detected by the pair of electrocardiographic electrodes 15 and 15 and photoplethysmographic signals detected by the photoplethysmographic sensors 10 and 20 are read in step S100. In step S102, filtering processing is performed on the electrocardiographic signals and photoplethysmographic signals read in step S100. Furthermore, second-order derivative is preferably performed on the photoplethysmographic signals, and acceleration pulse waves are thus acquired.

Then, in step S104, for example, the wearing state of the pulse wave propagation time measuring device 1 is determined based on the light reception amount of the photoplethysmographic sensors 10 and 20. That is, in the photoplethysmographic sensors 10 and 20, light applied from the light emitting elements 101 and 201, transmitting through a living body or reflected by the living body, and returning is received at the light reception elements 102 and 202, and a change in the amount of light is detected as a photoplethysmographic signal. In a state in which the device is not worn properly, the light reception amount of signal light decreases. Thus, in step S104, it is determined whether or not the light reception amount is equal to or more than a threshold (specific) value. In the case where the light reception amount is equal to or more than the threshold value, the process proceeds to step S108. In contrast, in the case where the light reception amount is less than the threshold value, it is determined that the device is not properly worn. Then, in step S106, device error information (warning information) is output. Thereafter, the process once exits from the main processing procedure. In place of the above-described method in which the light reception amount of the photoplethysmographic sensor 20 is used, a method in which the amplitude of a photoplethysmographic signal, the degree of stability of a baseline of an electrocardiographic waveform, a noise frequency component ratio, or other methods may be adopted.

In step S108, it is determined whether or not the acceleration of the neck region detected by the acceleration sensor 22 is equal to or more than a specific threshold (that is, whether or not the neck region moves and body motion noise increases). In the case where the acceleration of the neck region is less than the specific threshold, the process proceeds to step S112. In contrast, in the case where the acceleration of the neck region is equal to or more than the specific threshold, body motion error information is output in step S110, and the process exits from the main processing procedure.

In step S112, based on 3-axis acceleration data, the posture of the user (measurement part) is determined. In next step S114, the peak of the electrocardiographic signals and the peaks of the photoplethysmographic signals (acceleration pulse wave signals) are detected. Then, a time difference between the R wave peak of the detected electrocardiographic signals and the peak of each of the two photoplethysmographic signals (acceleration pulse waves) is calculated.

Next, in step S116, the delay times (deviation amounts) of the R wave peak of the electrocardiographic signals and the peaks of the photoplethysmographic signals (acceleration pulse waves) are obtained, and the time difference (peak time difference) between the R wave peak of the electrocardiographic signals and the peaks of the photoplethysmographic signals (acceleration pulse waves) is corrected, based on the obtained delay times.

Then, in step S118, it is determined whether or not the peak time difference corrected in step S116 is within a specific time (for example, 0.01 seconds or more and 0.3 seconds or less). In the case where the peak time difference is within the specific time, the process proceeds to step S122. In contrast, in the case where the peak time difference is outside the specific time, error information (noise determination) is output in step S120, and then the process once exits from the main processing procedure.

In step S122, pressure information is read from the pressure sensor 23. Next, in step S124, it is determined whether or not a pulse wave propagation time has been stabilized. In the case where the pulse wave propagation time has been stabilized, the process proceeds to step S134. In contrast, in the case where the pulse wave propagation time has not been stabilized, the process proceeds to step S126.

In step S126, it is determined whether or not pressure is appropriate (e.g., within a specific range). In the case where the pressure is not appropriate, the pressure is adjusted in step S128, and then the process proceeds to step S130. In contrast, the pressure is appropriate, the pressure is maintained, and the process proceeds to step S130.

In step S130, the heartbeat interval, pulse interval, and the like are determined. After that, the determined data is output in step S132, and the process exits from the main processing procedure.

In the case where a stable pulse wave propagation time is obtained, in step S134, the heartbeat interval, the pulse interval, a pulse wave propagation time, a temporal change of the pulse wave propagation time, and the like are determined. Then, in step S136, a constant of an estimation expression (conversion expression) for blood pressure is determined. Then, in step S138, estimation of the blood pressure of an artery, estimation of the status of blood pressure of an arteriole or a capillary, measurement of circulatory dynamics, and the like are performed. A method for estimating the status of blood pressure and circulatory dynamics is described above, and therefore, detailed explanation will be omitted here. Then, in step S140, the acquired blood pressure status, circulatory dynamics, and the like are output to, for example, a memory or an external apparatus such as a smartphone. Then, the process exits from the main processing procedure.

As described above, according to this embodiment, circulatory dynamics including the status of blood pressure of an arteriole or a capillary can be measured on the basis of a temporal change of a pulse wave propagation time acquired from a photoplethysmographic signal of the arteriole or the capillary after measurement is started (beginning of measurement). At this time, for example, a pulse wave propagation time between the arteriole or the capillary and an artery near the arteriole or the capillary is measured, and a temporal change of the pulse wave propagation time at the beginning of the measurement is measured. Therefore, a blood pressure difference between the arteriole or the capillary and the artery is measured, and circulatory dynamics can be measured. As a result, circulatory dynamics including the status of blood pressure of the arteriole or the capillary can be measured more simply and more accurately.

In particular, according to this embodiment, a pulse wave propagation time between an arteriole or a capillary and a (nearby) artery from which the arteriole or the capillary branches off can be measured, and a temporal change of the pulse wave propagation time at the beginning of measurement can be measured. Therefore, a blood pressure difference between the artery and the arteriole or the capillary can be measured, and circulatory dynamics can be measured. Furthermore, both pulse wave propagation times and the like can be measured at a single position. Therefore, blood pressure and the like of the arteriole or the capillary, including at the time of wearing, can be measured more simply. Unlike measurement at two separate points, blood pressure and the like of an arteriole or a capillary can be measured genuinely (that is, accurately).

According to this embodiment, by measuring the pulse wave propagation time between a carotid artery and an arteriole or a capillary near the carotid artery and measuring a temporal change of the pulse wave propagation time, a difference in blood pressure between the carotid artery and the arteriole or the capillary can be measured, and circulatory dynamics can be measured. When a pulse wave propagation time is measured at an arteriole or a capillary near a carotid artery (wide artery), it takes a time corresponding to the length of the arteriole or the capillary branching off from the carotid artery for arrival of a pulse wave. Therefore, the pulse wave propagation time is larger than the value measured at the carotid artery. Furthermore, in particular, blood pressure and circulatory dynamics of an arteriole or a capillary near a carotid artery out of arteries that may be measured relate to cardiovascular diseases, in particular, stroke. Therefore, estimation of blood pressure of an arteriole or a capillary near a carotid artery and observation of circulatory dynamics may be used for estimation of the risk of cardiovascular diseases.

Light of a visible light region (in particular, blue to yellow-green with a wavelength of 600 nm or less) is easily absorbed by a living body, unlike near-infrared light. Therefore, with the use of a light source (photoplethysmographic sensor) of a visible light region as the light emitting element 201 (second photoplethysmographic sensor 20), it is difficult for light to reach arteries underneath the skin. Therefore, even if the second photoplethysmographic sensor 20 is arranged immediately above an artery, a pulse wave signal of an arteriole or a capillary (not a pulse wave signal of an artery) can be obtained. In contrast, with the use of the first photoplethysmographic sensor 10 including the light emitting element 101 that outputs near-infrared light that is relatively difficult to be absorbed by a living body, a pulse wave signal of a carotid artery can be obtained. Therefore, according to this embodiment, in the vicinity of an artery, a photoplethysmographic signal corresponding to the blood flow of an arteriole or a capillary and a photoplethysmographic signal corresponding to the blood flow of a wider artery can be obtained at the same time (that is, pulse wave propagation time can be obtained).

According to this embodiment, by measuring an electrocardiogram (in particular, an R wave) at the same time as a reference, in addition to the pulse wave propagation time between a carotid artery and an arteriole or a capillary near the carotid artery, the pulse wave propagation time between the heart and the carotid artery and the pulse wave propagation time between the heart and the arteriole or the capillary can be obtained. Therefore, blood pressure at the artery can be estimated, and together with the estimated blood pressure of the arteriole or the capillary, evaluation of circulatory dynamics and the like can be performed more accurately.

According to this embodiment, posture of a user is detected, and a temporal change of the acquired pulse wave propagation time after measurement is started is acquired in accordance with the detected posture (that is, in consideration of the posture). Thus, a temporal change and the like of the pulse wave propagation time can be measured in a stable manner, without being affected by a change of posture.

According to this embodiment, a reference posture is set from among detected postures, and a temporal change of a pulse wave propagation time after measurement is started is obtained on the basis of time-series data of the pulse wave propagation time for the reference posture. Therefore, a temporal change and the like of the pulse wave propagation time can be measured in a stable manner, without being affected by a change of posture.

According to this embodiment, a reference posture is set from among detected postures, and time-series data of a pulse wave propagation time classified into a posture different from the reference posture is corrected in accordance with the reference posture. In addition, a temporal change of a pulse wave propagation time after measurement is started is obtained on the basis of time-series data of a pulse wave propagation time of the reference posture and the corrected time-series data of the pulse wave propagation time. Therefore, a temporal change and the like of the pulse wave propagation time can be measured in a stable manner, without being affected by a change of posture.

According to this embodiment, by measuring a temporal change of pulse wave propagation times for multiple postures after measurement is started, for example, a difference in blood pressure between arterioles or capillaries vertically lower and upper than an artery from which the arterioles or the capillaries branch off from can be measured, and circulatory dynamics can be observed more accurately.

According to this embodiment, pressures of the photoplethysmographic sensors 10 and 20 are measured, and a constant of a conversion expression of a pulse wave propagation time and blood pressure of an arteriole or a capillary is changed according to the pressures. Therefore, the accuracy of estimation of blood pressure and evaluation of circulatory dynamics of an arteriole or a capillary can be improved. Furthermore, according to this embodiment, a mechanism for adjusting pressure according to the measured pressure is provided. Therefore, an optimal pressure can be maintained, and the accuracy of estimation of blood pressure and the like can thus be improved.

Embodiments of the present invention have been described in detail above. However, the present invention is not limited to the embodiments described above, and various modifications may also be made to the present invention. For example, in an embodiment described above, the blood pressure status measuring apparatus 3 of a neck band type that holds the neck region of a user with the neck band 13 has been explained as an example. However, a mode in which a blood pressure status measuring apparatus that is attached along the back of the neck region of a user from one lateral side to the other lateral side of the neck region of the user and used is also possible. Furthermore, instead of a mode in which a blood pressure status measuring apparatus is worn on the neck region (neck) of a user, an electrocardiographic electrode, a pulse wave sensor, and a 3-axis acceleration sensor may be, for example, attached at an axillary part where an axillary artery exists, of a wrist watch type that is worn on a wrist where a radial artery exists, or of a sock type that is worn on a foot where a dorsalis pedis artery or a posterior tibial artery exists.

In the embodiments described above, for estimation of a change in blood pressure based on a change of a pulse wave propagation time, a predetermined correlation expression of a pulse wave propagation time and blood pressure is used. However, a conversion table in which the relationship of the pulse wave propagation time and blood pressure is defined for each posture may be used, instead of the correlation expression.

In the embodiments described above, a photoplethysmographic sensor using near-infrared light as the first photoplethysmographic sensor 10 that acquires a pulse wave signal of a carotid artery is used. However, in place of the photoplethysmographic sensor, for example, a piezoelectric pulse wave sensor, an electrocardiographic sensor (electrocardiographic electrode), or the like may be used. Furthermore, in addition to the various sensors described above, for example, a biological sensor such as an oxygen saturation sensor, a sound sensor (microphone), a displacement sensor, a temperature sensor, a humidity sensor, or the like may be used.

In the embodiments described above, processing including posture determination, correction of a pulse wave propagation time for each posture, estimation of the status of blood pressure (circulatory dynamics), and the like is performed by the signal processor 31. However, the acquired electrocardiographic signal, photoplethysmographic signal, and data such as 3-axis acceleration or the like may be wirelessly output to, for example, a personal computer (PC) or a smartphone, and processing such as the above-mentioned posture determination, correction of a pulse wave propagation time for each posture, estimation of a blood pressure status (circulatory dynamics), and the like may be performed on a PC or smartphone side. In this case, data of the correlation expression described above and the like are stored in a PC or smartphone side.

Furthermore, input means for receiving an operation for inputting a value of the height or sitting height of a user may further be provided. With the use of a correlation expression of a predetermined height or sitting height and an arterial length between an aortic valve and a neck region (carotid artery), the arterial length between the aortic valve and the neck region (carotid artery) may be obtained on the basis of the height or setting height value of the user and a blood pressure value of an arteriole or a capillary may be corrected in accordance with the arterial length. In this case, based on the received value of the height or sitting height of the user, the arterial length between the aortic valve and the neck region (carotid artery) is obtained, and a blood pressure value of the arteriole or the capillary is corrected in accordance with the arterial length. Therefore, accuracy of estimation of blood pressure and the like may be further improved.

In the embodiments described above, three pulse wave propagation times are obtained by a combination of a first photoplethysmographic signal, a second photoplethysmographic signal, and an electrocardiographic signal. However, any one (or two) of the pulse wave propagation times may be acquired to estimate blood pressure and the like.

### REFERENCE SIGNS LIST

1: pulse wave propagation time measuring device
3: blood pressure status measuring apparatus
11 and 12: sensor unit
13: neck band
15: electrocardiographic electrode
10: first photoplethysmographic sensor
101: first light emitting element
102: first light receiving element
20: second photoplethysmographic sensor
201: second light emitting element
202: second light receiving element
22: acceleration sensor
23: pressure sensor
31: signal processor
310: first signal processing unit
320: second signal processing unit
339: third signal processing unit
311, 321 and 331: amplification unit
312, 322, and 332: analog filter
313, 323, and 333: A/D converter
314, 324, and 334: digital filter
315 and 325: second-order derivative processing unit
316, 326, and 336: peak detection unit
318, 328, and 338: peak correction unit
330: pulse wave propagation time measuring unit
340: posture classification unit
341: pulse wave propagation time change acquisition unit (change acquiring means)
342: blood pressure status measuring unit (measuring means)
60: wireless communication module
70: pressure adjustment mechanism

1. A blood pressure status measuring apparatus comprising:
a photoplethysmographic sensor that includes a light emitting element and a light receiving element and acquires a time varying photoplethysmographic signal of an arteriole or a capillary;
a biological sensor that acquires a time varying biological signal serving as a reference for measurement of a pulse wave propagation time;
pulse wave propagation time acquiring means for acquiring a pulse wave propagation time on the basis of the photoplethysmographic and biological signals;
change acquiring means for acquiring a temporal change of the pulse wave propagation time acquired by the pulse wave propagation time acquiring means after measurement is started; and
measuring means for measuring circulatory dynamics including a blood pressure status as a function of the temporal change of the pulse wave propagation time after the measurement is started.

2. The blood pressure status measuring apparatus according to claim 1, wherein:
the biological sensor is a pulse wave detector which acquires a pulse wave signal of an artery from which the arteriole or the capillary branches off; and
the pulse wave propagation time acquiring means acquires the pulse wave propagation time on the basis of the photoplethysmographic signal of the arteriole or the capillary and the pulse wave signal of the artery.

3. The blood pressure status measuring apparatus according to claim 2, wherein:
the light emitting element outputs blue to yellow-green light; and
the pulse wave detecting means is a photoplethysmographic sensor that includes a light emitting element outputting near-infrared light.

4. The blood pressure status measuring apparatus according to claim 2, wherein:
the light emitting element outputs blue to yellow-green light; and
the pulse wave detecting means is a piezoelectric pulse wave sensor that acquires a piezoelectric pulse wave signal.

5. The blood pressure status measuring apparatus according to claim 1, wherein:
the biological sensor is a sensor that acquires an electrocardiographic signal; and
the pulse wave propagation time acquiring means acquires a pulse wave propagation time on the basis of the photoplethysmographic signal of the arteriole or the capillary acquired by the photoplethysmographic sensor and an R wave of the electrocardiographic signal acquired by the biological sensor.

6. The blood pressure status measuring apparatus according to claim 5, further comprising an electrocardiographic electrode that acquires an electrocardiographic signal, and wherein the pulse wave propagation time acquiring means also acquires, in addition to the pulse wave propagation time based on the photoplethysmographic signal of the arteriole or the capillary acquired by the photoplethysmographic sensor and the pulse wave signal of the artery acquired by the pulse wave detecting means, a pulse wave propagation time based on the photoplethysmographic signal of the arteriole or the capillary acquired by the photoplethysmographic sensor and the R wave of the electrocardiographic signal acquired by the electrocardiographic electrode and a pulse wave propagation time based on the pulse wave signal of the artery acquired by the pulse wave detecting means and the R wave of the electrocardiographic signal acquired by the electrocardiographic electrode.

7. The blood pressure status measuring apparatus according to claim 1, wherein:
the biological sensor is heart sound acquiring sensor for acquiring a heart sound signal; and
the pulse wave propagation time acquiring means acquires the pulse wave propagation time as a function of the photoplethysmographic signal of the arteriole or the capillary and the heart sound signal.

8. The blood pressure status measuring apparatus according to claim 2, further comprising a heart sound sensor for acquiring a heart sound signal, and wherein the pulse wave propagation time acquiring means acquires, in addition to the pulse wave propagation time based on the photoplethysmographic signal of the arteriole or the capillary acquired by the photoplethysmographic sensor and the pulse wave signal of the artery acquired by the pulse wave detecting means, a pulse wave propagation time based on the photoplethysmographic signal of the arteriole or the capillary acquired by the photoplethysmographic sensor and the heart sound signal acquired by the heart sound acquiring means and a pulse wave propagation time based on the pulse wave signal of the artery acquired by the pulse wave detecting means and the heart sound signal acquired by the heart sound acquiring means.

9. The blood pressure status measuring apparatus according to claim 2, wherein the photoplethysmographic sensor acquires a photoplethysmographic signal of an arteriole or a capillary near a carotid artery.

10. The blood pressure status measuring apparatus according to claim 1, further comprising a posture detector for detecting a posture of a user at a time when a pulse wave propagation time is acquired by the pulse wave propagation time acquiring means, and wherein the change acquiring means acquires a temporal change of the acquired pulse wave propagation time after measurement is started, as a function of the posture detected by the posture detecting means.

11. The blood pressure status measuring apparatus according to claim 10, wherein the change acquiring means sets a reference posture from among detected postures, and obtains a temporal change of the pulse wave propagation time after measurement is started, on the basis of time-series data of the pulse wave propagation time of the reference posture.

12. The blood pressure status measuring apparatus according to claim 10, wherein the change acquiring means sets a reference posture from among detected postures, corrects, in accordance with the reference posture, time-series data of a pulse wave propagation time classified into a posture different from the reference posture, and obtains a temporal change of the pulse wave propagation time after measurement is started, on the basis of the time-series data of the pulse wave propagation time of the reference posture and the corrected time-series data of the pulse wave propagation time.

13. The blood pressure status measuring apparatus according to claim 10, wherein:
the change acquiring means obtains, for each of detected postures, a temporal change of the pulse wave propagation time after measurement is started; and
the measuring means measures circulatory dynamics including a blood pressure status on the basis of temporal changes of pulse wave propagation times after measurement is started for the individual postures.

14. The blood pressure status measuring apparatus according to claim 1, further comprising a pressure sensor for detecting pressure of the photoplethysmographic sensor, and wherein the measuring means changes a conversion expression to be used to calculate blood pressure of an arteriole or a capillary on the basis of a pulse wave propagation time, in accordance with the pressure detected by the pressure detecting means.

15. The blood pressure status measuring apparatus according to claim 14, further comprising a pressure adjustment mechanism for adjusting the pressure to a specific value in accordance with the pressure detected by the pressure detecting means.

16. The blood pressure status measuring apparatus according to claim 9, further comprising an input for receiving an operation for inputting a value of a height or a sitting height of a user, and wherein the measuring means obtains an arterial length between an aortic valve and a carotid artery on the basis of the value of the height or the sitting height received by the input, and corrects a blood pressure value of an arteriole or a capillary on the basis of the arterial length.

17. A blood pressure status measuring apparatus comprising:
a photoplethysmographic sensor that includes a light emitting element and a light receiving element and acquires a photoplethysmographic signal of an arteriole or a capillary which varies over time;
a biological sensor that acquires a biological signal that varies over time and that serves as a reference for measurement of a pulse wave propagation time;
one or more processors which are programmed to:
(a) acquire a pulse wave propagation time on the basis of the photoplethysmographic and biological signals;
(b) acquire a temporal change of the pulse wave propagation time after measurement is started; and
(c) measure circulatory dynamics including a blood pressure status as a function of the temporal change of the pulse wave propagation time after the measurement is started.

18. The blood pressure status measuring apparatus according to claim 17, wherein:
the biological sensor is a pulse wave detector which acquires a pulse wave signal of an artery from which the arteriole or the capillary branches off; and
the pulse wave propagation time is determined as a function of the photoplethysmographic signal of the arteriole or the capillary and the pulse wave signal of the artery.

19. The blood pressure status measuring apparatus according to claim 18, wherein:
the light emitting element outputs blue to yellow-green light; and
the pulse wave detecting means is a photoplethysmographic sensor that includes a light emitting element outputting near-infrared light.

20. The blood pressure status measuring apparatus according to claim 18, wherein:
the light emitting element outputs blue to yellow-green light; and
the pulse wave detecting means is a piezoelectric pulse wave sensor that acquires a piezoelectric pulse wave signal.
